(19) United States
(12) Patent Application Publication
(10) Pub. No.: US 2025/0171396 A1
(43) Pub. Date: May 29, 2025

(54) METHOD FOR CONTINUOUS-FLOW AMINATION OF ALKYL CARBOXYLIC ACID COMPOUND

(71) Applicants: Jiangxi Normal University, Nanchang (CN); Fudan University, Shanghai (CN)

(72) Inventors: Fener CHEN, Shanghai (CN); Xianjing ZHENG, Shanghai (CN); Chang LIU, Shanghai (CN); Yiqun TIAN, Shanghai (CN); Yajiao ZHANG, Shanghai (CN); Li WAN, Shanghai (CN); Chunxue PENG, Shanghai (CN); Songlin ZHENG, Shanghai (CN); Liangchuan LAI, Shanghai (CN)

(21) Appl. No.: 19/031,745

(22) Filed: Jan. 18, 2025

(30) Foreign Application Priority Data
Apr. 23, 2024 (CN) 202410488647.7

(51) Int. Cl. C07C 227/10; B01J 31/02; C07C 227/40

(52) U.S. Cl. CPC C07C 227/10; B01J 31/0237; C07C 227/40; C07C 2531/02

(57) ABSTRACT

A method for continuous-flow amination for an alkyl carboxylic acid compound is provided, in which an amination reagent and a catalyst are mixed in a first micro-mixer and then preheated in a preheater. The mixture is mixed with a substituted alkyl carboxylic acid solution in a second micro-mixer and reacted in a dynamic flow reactor to produce a mixture including a carboxyl-containing organic amine product. Then, the mixture is subjected to gas-liquid separation, and the liquid phase is collected and filtered to obtain a first filtrate and a first filter residue. The first filter residue is mixed with a base solution under stirring and filtered to obtain a second filtrate and a second filter residue. The second filter residue was dried to yield a high-purity carboxyl-containing organic amine product.

### CROSS-REFERENCE TO RELATED APPLICATIONS

This application claims the benefit of priority from Chinese Patent Application No. 202410488647.7, filed on Apr. 23, 2024. The content of the aforementioned application, including any intervening amendments made thereto, is incorporated herein by reference in its entirety.

### TECHNICAL FIELD

This application relates to organic chemical synthesis, and more particularly to a method for continuous-flow amination of an alkyl carboxylic acid compound.

### BACKGROUND

The Delepine reaction and the Gabriel reaction are commonly used for the preparation of primary amines. Although these reactions are carried out under mild conditions and can produce high-purity products, they suffer from poor atom economy, generating large amounts of chemical waste, which makes them challenging for industrial application. For example, in the Delepine reaction, hexamethylenetetramine serves as an amination reagent, but only one of its four nitrogen atoms is utilized, with the rest discharged as waste. Similarly, in the Gabriel reaction, phthalimide is used as an ammonia source, producing phthalic acid as a byproduct, which is also difficult to recycle. Therefore, it is important to develop a catalytic amination system to address issues such as high reagent consumption and severe pollution in primary amine synthesis.

Chinese patent publication NO. 105037186A discloses a catalytic ammonolysis reaction. Although a catalyst is employed to achieve ammonolysis and a nitrogen source is recycled, the recovery of the catalyst and the purification of the aminated products are not addressed. Meanwhile, the reaction is performed using a traditional batch process, resulting in low production efficiency and prolonged reaction time. Chinese patent publication NO. 102241600A discloses a similar catalytic ammonolysis reaction. However, the recovery of both the catalyst and the nitrogen source is not addressed. During purification, water is added to separate components based on differences in solubility, resulting in significant loss of the product, which is dissolved in water. Furthermore, large quantities of wastewater containing organic compounds are generated, leading to severe environmental pollution and making the process unsuitable for industrial applications. Chinese patent publication NO. 102816077A employs water as a solvent, which similarly results in significant product dissolution into the water, leading to a reduced yield. Additionally, large quantities of wastewater containing organic compounds are generated, causing severe environmental pollution and making the process unsuitable for industrial applications.

In this regard, there is an urgent need to develop a novel amination process with high atom economy, minimal pollution, recyclable catalysts and high product separation yield. Additionally, this process can also solve the problems of poor continuity, limited scalability and low production efficiency in traditional batch production.

### SUMMARY

An object of the disclosure is to provide a method for continuous-flow amination of an alkyl carboxylic acid compound to overcome the defects in the prior art, including prolonged reaction time, low separation yield, non-recyclable catalyst, high waste emission, excessive energy consumption and poor product quality in the amination of alkyl carboxylic acids.

Technical solutions of the present disclosure are described as follows.

A method for continuous-flow amination of an alkyl carboxylic acid compound by using a fully continuous-flow synthesis system, the fully continuous-flow synthesis system comprising a first micro-mixer, a second micro-mixer, a dynamic flow reactor, a third micro-mixer and a microchannel reactor communicated in sequence, and the method comprising:

(1) mixing an amination reagent (I) with a solution of a catalyst in the first micro-mixer to obtain a first mixture; preheating the first mixture in a first preheater; mixing the first mixture with a solution of a substituted alkyl carboxylic acid (II) in the second micro-mixer to obtain a second mixture; reacting the second mixture in the dynamic flow reactor (a microchannel reactor with a special structure) to produce a first reaction mixture comprising a carboxyl-containing organic amine compound (III), as shown in the following reaction scheme:

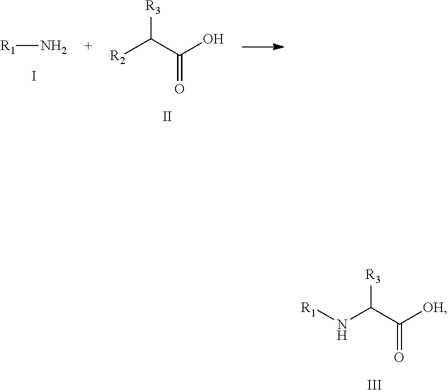

wherein a reaction pressure is controlled by a back-pressure valve;

subjecting the first reaction mixture to gas-liquid separation in a gas-liquid separator to collect a solid-containing liquid phase; filtering the solid-containing liquid phase in a first filtration-stirring tank to obtain a first filtrate and a first filter residue;

transferring the first filtrate to a first storage tank through a first three-way valve; mixing the first filter residue with a solution of a base under stirring followed by filtration to obtain a second filtrate and a second filter residue;

transferring the second filtrate to a second storage tank through a second three-way valve; and drying the second filter residue to obtain the carboxyl-containing organic amine compound (III);

(2) pumping the first filtrate from the first storage tank to a second preheater followed by preheating; concentrating the first filtrate in a nitrogen gas flow in an online evaporator to obtain an concentrated product, wherein a rate of the nitrogen gas flow is controlled by using a flowmeter such that a concentration of the catalyst in the concentrated product is the same as that in the solution of the catalyst in step (1), and the concentrated product is suitable as the solution of the catalyst for a next synthesis process; and condensing an evaporated solvent in a condenser followed by transfer to a first collection tank for recovery; and

(3) pumping the second filtrate from the second storage tank to the third micro-mixer followed by mixing with ammonia source to obtain a third mixture; reacting the third mixture in the microchannel reactor to obtain a second reaction mixture; filtering the second reaction mixture in a second filtration-stirring tank to obtain a third filtrate and a third filter residue; and transferring the third filtrate to a third storage tank for recovery; wherein the third filtrate is suitable as the solution of the base in the next synthesis process, and the third filter residue is a co-produced ammonium salt.

In some embodiments, in step (1), the amination reagent (I) is selected from the group consisting of ammonia gas, liquid ammonia, methylamine, ethylamine and benzylamine; the substituted alkyl carboxylic acid (II) is selected from the group consisting of fluoroacetic acid, chloroacetic acid, bromoacetic acid and iodoacetic acid.

In some embodiments, in step (1), the amination reagent is solvent-free or dissolved in methanol; a solvent in the solution of the catalyst is selected from the group consisting of methanol, ethanol, isopropanol and acetone; and a solvent in the solution of the substituted alkyl carboxylic acid (II) is methanol, ethanol or acetone.

In some embodiments, in step (1), the catalyst is amantadine or hexamethylenetetramine.

In some embodiments, in step (1), a molar ratio of the amination reagent (I) to the substituted alkyl carboxylic acid (II) is 1.2-3.5:1, a concentration of the amination reagent (I) is 90-99 wt. %, and a molar ratio of the catalyst to the substituted alkyl carboxylic acid (II) is 0.05-0.45:1, preferably 0.1-0.40:1.

In some embodiments, in step (1), the first mixture is preheated in the first preheater at 30-50° C. for 3-5 min; and the second mixture is reacted in the dynamic flow reactor at 70-100°° C. under a back pressure of 5-15 bar for 10-180 min, preferably 20-150 min.

In some embodiments, in step (1), the first filter residue is mixed, stirred with the solution of the base at 55-110° C. for 30-150 min, preferably at 55-80°° C. for 30-120 min.

In some embodiments, the base is selected from the group consisting of methylamine, ethylamine, butylamine, trimethylamine, triethylamine, tributylamine, N,N-diisopropylethylamine, pyridine, 4-dimethylaminopyridine and a combination thereof; and a molar ratio of the base to the substituted alkyl carboxylic acid (II) is 1.5-4.0:1.

In some embodiments, in step (2), a temperature of the second preheater is 30-120° C.; the online evaporator is a jacket heat exchanger, and a temperature of a heat exchange fluid in the jacket heat exchanger is 45-100° C.; and a flow rate ratio of nitrogen gas to the first filtrate is 3-15:1.

In some embodiments, in step (3), the third mixture is reacted in the microchannel reactor at 30-50°° C. under a pressure of 1-5 bar for 2-20 min, preferably reacted for 5-15 min.

In some embodiments, the first micro-mixer, the second micro-mixer and the third micro-mixer are each independently a plate-type micro-mixer having an inner diameter of 0.6-4.5 mm and a length of 2.5-45 m.

In some embodiments, in step (1), the dynamic flow reactor is a heat exchange jacket-equipped horizontal or vertical multi-stage rotating stirring reactor; and
the dynamic flow reactor comprises:
a cylindrical cavity;
wherein a wall of the cylindrical cavity is configured as a heat exchange fluid interlayer;
the cylindrical cavity is provided with a central shaft; the central shaft is provided with a plurality of stirring paddles for enhancing mass and heat transfer; the central shaft is configured to be driven by a motor to rotate at 50-500 rpm; a first end of the cylindrical cavity is provided with a reaction product inlet, and a second end of the cylindrical cavity is provided with a reaction product outlet; a first end of the heat exchange fluid interlayer is provided with a heat exchange fluid inlet, and a second end of the heat exchange fluid interlayer is provided with a heat exchange fluid outlet; and the cylindrical cavity has an inner diameter of 10-300 mm and a length of 2.5-30 m, as shown in FIG. 2.

In some embodiments, the first preheater and the second preheater each have a tubular microchannel structure having an inner diameter of 0.8-45 mm and a length of 5-1000 m.

In some embodiments, in steps (1) and (3), the microchannel reactor is made of a material selected from the group consisting of polytetrafluoroethylene, polyvinylidene fluoride, stainless steel, Hastelloy, zirconium, tantalum, nickel, silicon carbide, glass and a combination thereof.

Compared to the prior art, the present disclosure has the following beneficial effects.

(1) The micro-mixer can significantly enhance the mass transfer effect of multiphase systems, increase the reaction rate and reduce the reactor volume. Meanwhile, the microchannel reactor is characterized by excellent mass transfer, heat transfer and continuous material mixing performance, effectively shortening the reaction time, improving reaction efficiency, and increasing the throughput per unit reactor volume. The microchannel reactor also ensures higher reaction safety and significantly reduces waste emissions and energy consumption. The reaction time is reduced from 5-10 h in traditional batch reactors to just a few minutes for target product preparation.

(2) The method provided herein enables catalyst recycling, reduces production costs, increases yields and effectively lowers waste emissions.

(3) The amination reaction is carried out in the dynamic flow reactor, which provides sufficient stirring and excellent heat transfer, enhancing reaction safety while reducing the consumption of amination reagents and energy and making the process more environmentally friendly.

(4) The method disclosed herein achieves continuous synthesis from raw materials to the desired product, with an uninterrupted process and a high degree of automation. No external intervention is required during intermediate steps, resulting in high spatiotemporal efficiency, significantly reducing labor requirements and intensity, and substantially lowering production costs.

(5) The method provided herein achieves continuous production of the desired product, with a conversion rate of over 99%, a yield of over 95% and a purity of over 99.5%.

(6) The microchannel reactor employed herein enables the convenient industrial-scale production of the disclosed method through the strategies of multi-channel parallel scaling or size scaling.

### BRIEF DESCRIPTION OF THE DRAWINGS

FIG. 1 is a flow chart of a method for continuous-flow amination of an alkyl carboxylic acid compound according to an embodiment of the present disclosure; and

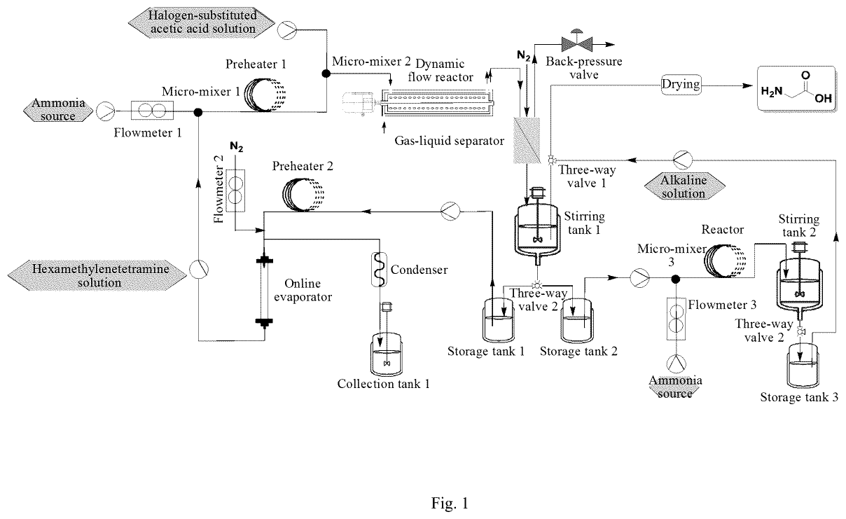

FIG. 2 schematically shows a dynamic flow reactor according to an embodiment of the present disclosure.

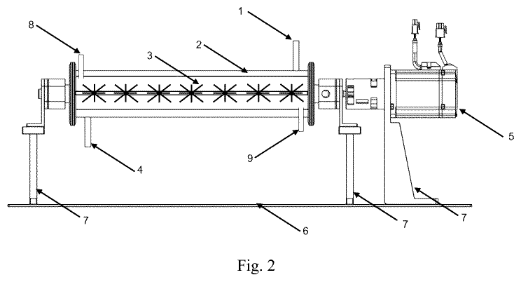

In the figures: 1-heat exchange fluid outlet; 2-heat exchange fluid interlayer; 3-coaxial multistirring-paddle; 4-heat exchange fluid inlet; 5-motor; 6-base plate; 7-support frame; 8-reaction product outlet; and 9-reaction product inlet.

### DETAILED DESCRIPTION OF EMBODIMENTS

The present disclosure will be described in detail below with reference to embodiments. It should be noted that the described embodiments are merely illustrative, and are not intended to limit the disclosure.

### Example 1

The methanol solution of hexamethylenetetramine (0.40 equivalent) was mixed with ammonia gas in a first Y-type micro-mixer to obtain a first mixture. The methanol solution of hexamethylenetetramine was pumped at a flow rate of 3.1 mL/min by a first feed pump. A flow rate of ammonia gas was precisely controlled to 10 sccm using a flowmeter. Then, the first mixture was introduced into a first preheated reactor for reaction at 35° C. for 3 min to obtain a preheated mixture. The first preheated reactor had an inner diameter of 5 mm and a length of 100 m. The preheated mixture and a methanol solution of chloroacetic acid (1.0 equivalent) were pumped into a second Y-type micro-mixer at a flow rate of 1.1 mL/min by a second feed pump to obtain a second mixture. Then, the second mixture was subjected to an amination reaction in a dynamic flow reactor at 90° C. for 40 min to obtain a third mixture. The dynamic flow reactor had an inner diameter of 150 mm and a length of 2.0 m. A pressure of a back-pressure valve was 8 atm. The third mixture was filtered in a first gas-liquid separator to obtain a first solid-containing liquid phase. The first solid-containing liquid phase was introduced into a first filtration-stirring tank followed by a first filtration to obtain a first filtrate and a first filter residue. The first filter residue was the desired crude product.

The desired crude product was stirred in a first filtration-stirring tank reactor with a methanol solution of triethylamine (1.0 equivalent) at 65° C. for 80 min to obtain a fourth mixture. The methanol solution of triethylamine was pumped in at a flow rate of 2.1 mL/min by a third feed pump. Then, the fourth mixture was subjected to a second filtration to obtain a second filtrate and a second filter residue. The second filter residue was dried to obtain the desired high-purity product with a yield of 95% and a purity of 99.8%.

The first filtrate was mixed with nitrogen gas in a micro-mixer to obtain a fifth mixture. The first filtrate was pumped at a flow rate of 4.0 mL/min using a fourth feed pump. A flow rate of nitrogen gas was controlled to 50 sccm by a flowmeter. Then, the fifth mixture was pumped into a second preheated reactor for reaction at 75° C. for 5 min. The second preheated reactor had an inner diameter of 5 mm and a length of 30 m. A temperature of heat exchange fluid was 85° C. Methanol was concentrated in an online evaporator with a heat exchange jacket-equipped. The evaporated methanol was condensed in a condenser and collected in a collection tank. The concentrated methanol solution of hexamethylenetetramine was pumped into the first Y-type micro-mixer at a flow rate of 3.1 mL/min by the first feed pump, and then introduced into the first preheated reactor for cyclic catalytic use.

The second filtrate was mixed with ammonia gas in a third Y-type micro-mixer to obtain a sixth mixture. The second filtrate was pumped at a flow rate of 2.1 mL/min using a fifth feed pump. A flow rate of ammonia gas was controlled to 10 sccm through a third flowmeter. Then, the sixth mixture was pumped into a microchannel reactor for reaction at 30° C. under a pressure of 1 bar for 5 min to obtain a seventh mixture. Then, the seventh mixture was filtered in a second gas-liquid separator to obtain a second solid-containing liquid phase. The second solid-containing liquid phase was introduced into a second filtration-stirring tank reactor followed by a third filtration to obtain a nitrogen fertilizer of ammonium chloride and the methanol solution of triethylamine. The methanol solution of triethylamine was then pumped at a flow rate of 2.1 mL/min by the third feed pump into the first filtration-stirring tank for cyclic purification and recovery.

### Example 2

A methanol solution of hexamethylenetetramine (0.30 equivalent) was mixed with ammonia gas in a first Y-type micro-mixer to obtain a first mixture. The methanol solution of hexamethylenetetramine was pumped at a flow rate of 5.1 mL/min by a first feed pump. A flow rate of ammonia gas was precisely controlled to 20 sccm using a flowmeter. Then, the first mixture was introduced into a first preheated reactor for reaction at 45° C. for 3 min to obtain a preheated mixture. The first preheated reactor had an inner diameter of 7 mm and a length of 200 m. The preheated mixture and a methanol solution of chloroacetic acid (1.3 equivalent) were pumped into a second Y-type micro-mixer at a flow rate of 1.5 mL/min by a second feed pump to obtain a second mixture. Then, the second mixture was subjected to an amination reaction in a dynamic flow reactor at 70°° C. for 50 min to obtain a third mixture. The dynamic flow reactor had an inner diameter of 200 mm and a length of 2.5 m. A pressure of a back-pressure valve was 10 atm. The third mixture was filtered in a first gas-liquid separator to obtain a first solid-containing liquid phase. The first solid-containing liquid phase was introduced into a first filtration-stirring tank followed by a first filtration to obtain a first filtrate and a first filter residue. The first filter residue was the desired crude product.

The desired crude product was stirred in a first filtration-stirring tank with a methanol solution of triethylamine (1.2 equivalent) at 70°° C. for 70 min to obtain a fourth mixture. The methanol solution of triethylamine was pumped in at a flow rate of 4.0 mL/min by a third feed pump. Then, the fourth mixture was subjected to a second filtration to obtain a second filtrate and a second filter residue. The second filter residue was dried to obtain the desired high-purity product with a yield of 95% and a purity of 99.5%.

The first filtrate was mixed with nitrogen gas in a micro-mixer to obtain a fifth mixture. The first filtrate was pumped at a flow rate of 6.5 mL/min using a fourth feed pump. A flow rate of nitrogen gas was controlled to 100 sccm by a flowmeter. Then, the fifth mixture was pumped into a second preheated reactor for reaction at 80° C. for 4 min. The second preheated reactor had an inner diameter of 5 mm and a length of 25 m. A temperature of heat exchange fluid was 90° C. Methanol was concentrated in an online evaporator with a heat exchange jacket-equipped. The evaporated methanol was condensed in a condenser and collected in a collection tank. The concentrated methanol solution of hexamethylenetetramine was pumped into the first Y-type micro-mixer at a flow rate of 5.1 mL/min by the first feed pump, and then introduced into the first preheated reactor for cyclic catalytic use.

The second filtrate was mixed with ammonia gas in a third Y-type micro-mixer to obtain a sixth mixture. The second filtrate was pumped at a flow rate of 4.0 mL/min using a fifth feed pump. A flow rate of ammonia gas was controlled to 20 sccm through a third flowmeter. Then, the sixth mixture was pumped into a microchannel reactor for reaction at 35° C. under a pressure of 3 bar for 5.5 min to obtain a seventh mixture. Then, the seventh mixture was filtered in a second gas-liquid separator to obtain a second solid-containing liquid phase. The second solid-containing liquid phase was introduced into a second filtration-stirring tank followed by a third filtration to obtain a nitrogen fertilizer of ammonium chloride and the methanol solution of triethylamine. The methanol solution of triethylamine was then pumped at a flow rate of 4.0 mL/min by the third feed pump into the first filtration-stirring tank for cyclic purification and recovery.

### Example 3

A methanol solution of hexamethylenetetramine (0.35 equivalent) was mixed with ammonia gas in a first Y-type micro-mixer to obtain a first mixture. The methanol solution of hexamethylenetetramine was pumped at a flow rate of 7.0 mL/min by a first feed pump. A flow rate of ammonia gas was precisely controlled to 30 sccm using a flowmeter. Then, the first mixture was introduced into a first preheated reactor for reaction at 55°° C. for 6.5 min to obtain a preheated mixture. The first preheated reactor had an inner diameter of 5 mm and a length of 250 m. The preheated mixture and a methanol solution of chloroacetic acid (1.8 equivalent) were pumped into a second Y-type micro-mixer at a flow rate of 3.2 mL/min by a second feed pump to obtain a second mixture. Then, the second mixture was subjected to an amination reaction in a dynamic flow reactor at 100° C. for 50 min to obtain a third mixture. The dynamic flow reactor had an inner diameter of 250 mm and a length of 3.0 m. A pressure of a back-pressure valve was 9 atm. The third mixture was filtered in a first gas-liquid separator to obtain a first solid-containing liquid phase. The first solid-containing liquid phase was introduced into a first filtration-stirring tank followed by a first filtration to obtain a first filtrate and a first filter residue. The first filter residue was the desired crude product.

The desired crude product was stirred in a first filtration-stirring tank with a methanol solution of triethylamine (1.2 equivalent) at 70° C. for 90 min to obtain a fourth mixture. The methanol solution of triethylamine was pumped in at a flow rate of 4.5mL/min by a third feed pump. Then, the fourth mixture was subjected to a second filtration to obtain a second filtrate and a second filter residue. The second filter residue was dried to obtain the desired high-purity product with a yield of 96% and a purity of 99.7%.

The first filtrate was mixed with nitrogen gas in a micro-mixer to obtain a fifth mixture. The first filtrate was pumped at a flow rate of 10 mL/min using a fourth feed pump. A flow rate of nitrogen gas was controlled to 250 sccm by a flowmeter. Then, the fifth mixture was pumped into a second preheated reactor for reaction at 80° C. for 8 min. The second preheated reactor had an inner diameter of 8 mm and a length of 50 m. A temperature of heat exchange fluid was 95° C. Methanol was concentrated in an online evaporator with a heat exchange jacket-equipped. The evaporated methanol was condensed in a condenser and collected in a collection tank. The concentrated methanol solution of hexamethylenetetramine was pumped into the first Y-type micro-mixer at a flow rate of 7.0 mL/min by the first feed pump, and then introduced into the first preheated reactor for cyclic catalytic use.

The second filtrate was mixed with ammonia gas in a third Y-type micro-mixer to obtain a sixth mixture. The second filtrate was pumped at a flow rate of 4.5 mL/min using a fifth feed pump. A flow rate of ammonia gas was controlled to 40 sccm through a third flowmeter. Then, the sixth mixture was pumped into a microchannel reactor for reaction at 35° C. under a pressure of 5 bar for 7 min to obtain a seventh mixture. Then, the seventh mixture was filtered in a second gas-liquid separator to obtain a second solid-containing liquid phase. The second solid-containing liquid phase was introduced into a second filtration-stirring tank equipped with a filtration function followed by a third filtration to obtain a nitrogen fertilizer of ammonium chloride and the methanol solution of triethylamine. The methanol solution of triethylamine was then pumped at a flow rate of 4.5 mL/min by the third feed pump into the first filtration-stirring tank for cyclic purification and recovery.

### Example 4

A methanol solution of hexamethylenetetramine (0.25 equivalent) was mixed with ammonia gas in a first Y-type micro-mixer to obtain a first mixture. The methanol solution of hexamethylenetetramine was pumped at a flow rate of 12.0 mL/min by a first feed pump. A flow rate of ammonia gas was precisely controlled to 50 sccm using a flowmeter. Then, the first mixture was introduced into a first preheated reactor for reaction at 40° C. for 5 min to obtain a preheated mixture. The first preheated reactor had an inner diameter of 10 mm and a length of 350 m. The preheated mixture and a methanol solution of chloroacetic acid (2.0 equivalent) were pumped into a second Y-type micro-mixer at a flow rate of 4.0 mL/min by a second feed pump to obtain a second mixture. Then, the second mixture was subjected to an amination reaction in a dynamic flow reactor at 110° C. for 60 min to obtain a third mixture. The dynamic flow reactor had an inner diameter of 300 mm and a length of 3.0 m. A pressure of a back-pressure valve was 10 atm. The third mixture was filtered in a first gas-liquid separator to obtain a first solid-containing liquid phase. The first solid-containing liquid phase was introduced into a first filtration-stirring tank followed by a first filtration to obtain a first filtrate and a first filter residue. The first filter residue was the desired crude product.

The desired crude product was stirred in a first filtration-stirring tank with a methanol solution of triethylamine (1.0 equivalent) at 70° C. for 85 min to obtain a fourth mixture. The methanol solution of triethylamine was pumped in at a flow rate of 6.0 mL/min by a third feed pump. Then, the fourth mixture was subjected to a second filtration to obtain a second filtrate and a second filter residue. The second filter residue was dried to obtain the desired high-purity product with a yield of 95% and a purity of 99.7%.

The first filtrate was mixed with nitrogen gas in a micro-mixer to obtain a fifth mixture. The first filtrate was pumped at a flow rate of 15.5 mL/min using a fourth feed pump. A flow rate of nitrogen gas was controlled to 100 sccm by a flowmeter. Then, the fifth mixture was pumped into a second preheated reactor for reaction at 85° C. for 5 min. The second preheated reactor had an inner diameter of 8 mm and a length of 55 m. A temperature of heat exchange fluid was 90° C. Methanol was concentrated in an online evaporator with a heat exchange jacket-equipped. The evaporated methanol was condensed in a condenser and collected in a collection tank. The concentrated methanol solution of hexamethylenetetramine was pumped into the first Y-type micro-mixer at a flow rate of 12.0 mL/min by the first feed pump, and then introduced into the first preheated reactor for cyclic catalytic use.

The second filtrate was mixed with ammonia gas in a third Y-type micro-mixer to obtain a sixth mixture. The second filtrate was pumped at a flow rate of 6.0 mL/min using a fifth feed pump. A flow rate of ammonia gas was controlled to 50 sccm through a third flowmeter. Then, the sixth mixture was pumped into a microchannel reactor for reaction at 35° C. under a pressure of 5 bar for 8 min to obtain a seventh mixture. Then, the seventh mixture was filtered in a second gas-liquid separator to obtain a second solid-containing liquid phase. The second solid-containing liquid phase was introduced into a second filtration-stirring tank followed by a third filtration to obtain a nitrogen fertilizer of ammonium chloride and the methanol solution of triethylamine. The methanol solution of triethylamine was then pumped at a flow rate of 6.0 mL/min by the third feed pump into the first filtration-stirring tank for cyclic purification and recovery.

Described above are merely preferred embodiments of the present disclosure, and are not intended to limit the scope of the present disclosure. It should be understood that various modifications, changes and replacements made by those skilled in the art without departing from the spirit of the disclosure shall fall within the scope of the present disclosure defined by the appended claims.

What is claimed is:

1. A method for continuous-flow amination of an alkyl carboxylic acid compound by using a fully continuous-flow synthesis system, the fully continuous-flow synthesis system comprising a first micro-mixer, a second micro-mixer, a dynamic flow reactor, a third micro-mixer and a microchannel reactor communicated in sequence, and the method comprising:
(1) mixing an amination reagent (I) with a solution of a catalyst in the first micro-mixer to obtain a first mixture; preheating the first mixture in a first preheater; mixing the first mixture with a solution of a substituted alkyl carboxylic acid (II) in the second micro-mixer to obtain a second mixture; reacting the second mixture in the dynamic flow reactor to produce a first reaction mixture comprising a carboxyl-containing organic amine compound (III), as shown in the following reaction scheme: 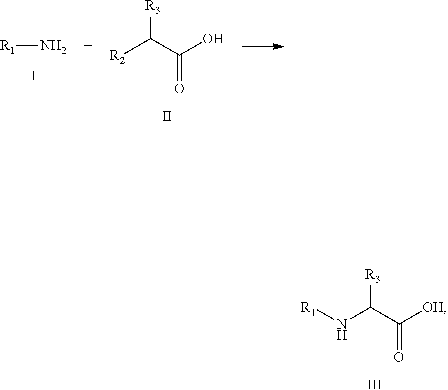
wherein a reaction pressure is controlled by a back-pressure valve;
subjecting the first reaction mixture to gas-liquid separation in a gas-liquid separator to collect a solid-containing liquid phase; filtering the solid-containing liquid phase in a first filtration-stirring tank to obtain a first filtrate and a first filter residue;
transferring the first filtrate to a first storage tank through a first three-way valve; mixing the first filter residue with a solution of a base under stirring followed by filtration to obtain a second filtrate and a second filter residue;
transferring the second filtrate to a second storage tank through a second three-way valve; and drying the second filter residue to obtain the carboxyl-containing organic amine compound (III);
(2) pumping the first filtrate from the first storage tank to a second preheater followed by preheating; concentrating the first filtrate in a nitrogen gas flow in an online evaporator to obtain an concentrated product, wherein a rate of the nitrogen gas flow is controlled by using a flowmeter such that a concentration of the catalyst in the concentrated product is the same as that in the solution of the catalyst in step (1), and the concentrated product is suitable as the solution of the catalyst for a next synthesis process; and condensing an evaporated solvent in a condenser followed by transfer to a first collection tank for recovery; and
(3) pumping the second filtrate from the second storage tank to the third micro-mixer followed by mixing with ammonia source to obtain a third mixture; reacting the third mixture in the microchannel reactor to obtain a second reaction mixture; filtering the second reaction mixture in a second filtration-stirring tank to obtain a third filtrate and a third filter residue; and transferring the third filtrate to a third storage tank for recovery; wherein the third filtrate is suitable as the solution of the base in the next synthesis process, and the third filter residue is a co-produced ammonium salt.

2. The method of claim 1, wherein in step (1), the amination reagent (I) is selected from the group consisting of ammonia gas, liquid ammonia, methylamine, ethylamine and benzylamine; the substituted alkyl carboxylic acid (II) is selected from the group consisting of fluoroacetic acid, chloroacetic acid, bromoacetic acid and iodoacetic acid; and the amination reagent is solvent-free or dissolved in methanol;
the catalyst is amantadine or hexamethylenetetramine; a solvent in the solution of the catalyst is selected from the group consisting of methanol, ethanol, isopropanol and acetone; and a solvent in the solution of the substituted alkyl carboxylic acid (II) is methanol, ethanol or acetone.

3. The method of claim 2, wherein in step (1), a molar ratio of the amination reagent (I) to the substituted alkyl carboxylic acid (II) is 1.2-3.5:1, a concentration of the amination reagent (I) is 90-99 wt. %, and a molar ratio of the catalyst to the substituted alkyl carboxylic acid (II) is 0.05-0.45:1;
the base is selected from the group consisting of methylamine, ethylamine, butylamine, trimethylamine, triethylamine, tributylamine, N,N-diisopropylethylamine, pyridine and 4-dimethylaminopyridine; and a molar ratio of the base to the substituted alkyl carboxylic acid (II) is 1.5-4.0:1.

4. The method of claim 1, wherein in step (1), the first mixture is preheated in the first preheater at 30-50° C. for 3-5 min; and the second mixture is reacted in the dynamic flow reactor at 70-100° C. under a back pressure of 5-15 bar for 10-180 min.

5. The method of claim 1, wherein in step (1), the first filter residue is mixed, with the solution of the base at 55-110° C. for 30-150 min.

6. The method of claim 1, wherein in step (2), a temperature of the second preheater is 30-120° C.; the online evaporator is a jacket heat exchanger, and a temperature of a heat exchange fluid in the jacket heat exchanger is 45-100° C.; and a flow rate ratio of nitrogen gas to the first filtrate is 3-15:1.

7. The method of claim 1, wherein in step (3), the third mixture is reacted in the microchannel reactor at 30-50° C. under a pressure of 1-5 bar for 2-20 min.

8. The method of claim 1, wherein the first micro-mixer, the second micro-mixer and the third micro-mixer are each independently a plate-type micro-mixer having an inner diameter of 0.6-4.5 mm and a length of 2.5-45 m.

9. The method of claim 1, wherein in step (1), the dynamic flow reactor is a heat exchange jacket-equipped horizontal or vertical multi-stage rotating stirring reactor; and
the dynamic flow reactor comprises:
a cylindrical cavity;
wherein a wall of the cylindrical cavity is configured as a heat exchange fluid interlayer;
the cylindrical cavity is provided with a central shaft; the central shaft is provided with a plurality of stirring paddles for enhancing mass and heat transfer; the central shaft is configured to be driven by a motor to rotate at 50-500 rpm; a first end of the cylindrical cavity is provided with a reaction product inlet, and a second end of the cylindrical cavity is provided with a reaction product outlet; a first end of the heat exchange fluid interlayer is provided with a heat exchange fluid inlet, and a second end of the heat exchange fluid interlayer is provided with a heat exchange fluid outlet; and he cylindrical cavity has an inner diameter of 10-300 mm and a length of 2.5-30 m.

10. The method of claim 1, wherein the first preheater and the second preheater each have a tubular microchannel structure having an inner diameter of 0.8-45 mm and a length of 5-1000 m.
